(19) United States
(12) Patent Application Publication
(10) Pub. No.: US 2021/0310027 A1
(43) Pub. Date: Oct. 7, 2021

(54) METHODS AND COMPOSITIONS FOR PRODUCING A VIRUS

(71) Applicant: OXFORD UNIVERSITY INNOVATION LIMITED, Oxfordshire (GB)

(72) Inventors: Sarah GILBERT, Oxfordshire (GB); Susan Jane MORRIS, Oxfordshire (GB)

(21) Appl. No.: 17/269,450

(22) PCT Filed: Aug. 30, 2019
(86) PCT No.: PCT/EP2019/073181

§ 371 Date: Feb. 18, 2021

(30) Foreign Application Priority Data
Aug. 30, 2018 (GB) 1814141.6

(51) Int. Cl. C12N 15/86; C12N 15/66; A61K 39/12; C12N 7/00

(52) U.S. Cl. CPC C12N 15/86; C12N 15/66; C12N 2710/10351; C12N 7/00; C12N 2710/10343; A61K 39/12

(57) ABSTRACT

The invention relates to methods for generating a recombinant adenovirus comprising a nucleotide sequence encoding a heterologous gene of interest for use as a vaccine comprising the steps of inserting the heterologous gene of interest into the adenovirus genome by recombining terminal protein complexed adenovirus genomic DNA (TPC-Ad gDNA) with a polynucleotide comprising a nucleotide sequence encoding the gene of interest and having 5′ and 3′ ends that are homologous to the insertion site sequence of the adenovirus genomic DNA in an in vitro recombination reaction, transfecting cells growing in individual vessels with a dilution of the in vitro recombination reaction mixture from (i) such that a number of such individual vessels contain a single cell that is infected by a recombinant adenovirus comprising the nucleotide sequence encoding the heterologous gene of interest, and identifying those individual vessels in which a single cell has been infected by the recombinant adenovirus comprising the nucleotide sequence encoding the heterologous gene of interest. Suitably said TPC-Ad gDNA comprises serotype-matched terminal protein and adenovirus genome, and said gene of interest codes for a single epitope, a string of epitopes, a segment of an antigen or a complete antigen protein. The invention also relates to recombinant adenoviruses and compositions made using these methods.

### FIELD OF THE INVENTION

The invention relates to rapid generation of recombinant adenoviruses for use in the induction of immune responses, suitably protective immune responses, against heterologous antigens including infectious pathogen antigens and tumour antigens associated with cancer.

### BACKGROUND TO THE INVENTION

Replication incompetent adenovirus vectors derived from either human serotype 5 adenovirus (HAdV-C5) or other human adenoviruses or simian adenoviruses have been used as vaccine vectors to deliver infectious pathogen antigens and cancer antigens in multiple clinical trials (Ewer et al. (2017) Hum Vaccin Immunother. 13(12):3020-3032; and Cappuccini et al. (2016) Cancer Immunol Immunother. 65(6):701-13.). These vectors offer a large number of advantages for vaccine development; they are not replication-competent in humans and therefore safer than replicating vectors; they infect replicating and non-replicating cells; they have a broad tissue tropism, they elicit high immune responses including particularly potent cellular immunity; and they are easily purified to high titres (Morris et al. (2016) Future Virology 11(9), 649-659). The advent of bacterial artificial chromosomes (BACs) coupled to bacteriophage X Red recombination (recombineering) technology has facilitated the cloning and manipulation of adenovirus genomes (Ruzsics Z., Lemnitzer F., Thirion C. (2014) Engineering Adenovirus Genome by Bacterial Artificial Chromosome (BAC) Technology. In: Chillón M., Bosch A. (eds) Adenovirus. Methods in Molecular Biology (Methods and Protocols), vol 1089. Humana Press, Totowa, N.J.). This technology coupled with recombination technology for the quick insertion of expression cassettes is currently used for the generation of adenoviral vectors. However, using this approach and traditional manufacturing processes the time taken from antigen identification to a clinical grade adenovirus vector is on average 33-44 weeks (FIG. 1). Preparation of a recombinant virus for use as a vaccine involves the generation of pre-GMP (pre-Good Manufacturing Practice) starting material that requires cloning of the recombinant virus after it is rescued following initial infection of permissive cells. This is a time consuming process and up to 3 rounds of cloning, each taking 5 weeks, are required due to the potential insertion of the chloramphenicol gene (used for bacterial artificial chromosome (BAC) selection in standard adenoviral genome manipulation processes) into the adenovirus genome and the heterogeneity of the viral vector produced from BAC-derived adenovirus genome. When using a BAC, any mutations in the adeno genome that may have been introduced during manipulation in bacteria will be carried over to the adenovirus. This problem is addressed in the method of the present invention in which the adenoviral genomic DNA has already been cloned and characterised before starting to generate a recombinant adenovirus and is therefore known to be correct. Consequently it is not necessary to sequence the adenoviral genomic DNA as that has been characterised previously.

Hillgenberg and co-workers (Journal of Virology 80(11) (2006) 5435-5450) and independently Choi and co-workers (Journal of Biotechnology 162 (2012) 246-252) have described rapid methods for generation of large volumes of recombinant adenoviruses using a recombineering approach that eliminated the need for shuttle vector construction, bacterial transformation and selection, and reduced effort required for plaque isolation. However, these authors sought to generate populations of recombinant adenoviruses expressing large numbers of different heterologous genes and failed to provide for raid and simple cloning of single recombinant viruses in their methods for use as vaccines. Single clones are required for clinical use of adenoviral vaccines under regulations related to GMP manufacturing and clinical use of such vaccines.

More recently Miciak and co-workers (PLoS ONE 13(6) (2018) e0199563) have described an in-vitro adenoviral genome assembly method from several fragments, which are then transfected into cells. These workers have also failed to produce a method that yields a cloned single recombinant virus in which it is not necessary to carry out a clonal selection method after the recombinant virus has been isolated.

Thus, there is no method in the prior art that is suitable for the rapid generation of recombinant adenoviruses for use as vaccines. The present invention seeks to address and overcome this challenge and overcome problem(s) associated with methods in the prior art by providing a new method for the generation of a small clinical grade batch of replication incompetent adenovirus vectors in under 4 weeks (FIG. 2).

### SUMMARY OF THE INVENTION

Adenoviruses are non-enveloped viruses with linear, double stranded DNA (dsDNA) genomes between 26-46 kb in length. Adenovirus genomic DNA is infectious when transfected into permissive cells as naked DNA. It has however, been reported that when human Ad-5 (HAdV-C5) genomic DNA (gDNA) complexed with the 55 kDa terminal protein (TP) from the same adenovirus is transfected into permissive cells 100-1000 fold more viral plaques are produced compared to naked DNA. The TP protects the viral gDNA from digestion by cellular exonucleases, acts as a primer for the initiation of DNA replication and forms a heterodimer with DNA polymerase. The DNA polymerase covalently couples the first dCTP with Ser-580 of HAdV-C5 TP. The human adenovirus TP enhances human adenovirus replication by increasing template activity over 20 fold compared to protein-free templates. This is through subtle changes in the origin of replication allowing binding of other replication factors. The TP also promotes transcription by mediating HAdV-C5 genomic DNA-host nuclear matrix association.

The present inventors sought to harness the property of increased plaque production from transfected TPC-adenoviral gDNA (TPC-Ad gDNA) in combination with existing recombination technology to generate clinical grade adenovirus vaccine vectors, with a focus on the now preferred simian adenoviral vectors.

TPC-Ad gDNA can be isolated and purified, tested for homogeneity and stored in advance of adenoviral production and manufacturing. This approach removes the need for propagation of adenoviral gDNA in bacteria and thus avoids the potential for insertion of the chloramphenicol gene (used for BAC selection) into the adenovirus genome and the potential for heterogeneity of the virus genome that can occur after multiple rounds of amplification in a bacterial host. Increased numbers of plaques generated after transfection of cells with TPC-Ad gDNA allows for successful rescue of recombinant virus when only a small number of recombinant adenoviral genomes are generated, and furthermore the resulting recombinant adenoviruses can be cloned quickly and easily at a very early stage in the manufacturing process. The present inventors have simplified the viral production and manufacturing process, and as a result remarkably they have made it possible to generate and manufacture a recombinant adenovirus for use as a vaccine in as little as 28 days. This shortening of the time for vaccine production will have many advantages which include i) allowing rapid generation of personalised cancer vaccines for treatment, by therapeutic immunisation, of malignancies more rapidly; ii) more rapid generation of vaccine against new outbreak pathogens in the face of a new epidemic, allowing manufacture and generation of larger quantities of vaccine more rapidly; and iii) a reduction in manufacturing costs in expensive GMP (good manufacturing practice) manufacturing facilities through a marked reduction of time in the facility.

In a first aspect, the invention provides a method for generating a recombinant adenovirus comprising a nucleotide sequence encoding a heterologous gene of interest for use as a vaccine comprising the steps of: (i) inserting the heterologous gene of interest into the adenovirus genome by recombining terminal protein complexed adenovirus genomic DNA (TPC-Ad gDNA) with a synthetic DNA comprising a nucleotide sequence encoding the gene of interest and having at least 15 bp at its 5′ end and at least 15 bp at its 3′ end that are homologous to the insertion site sequence of the adenovirus genomic DNA in an in vitro recombination reaction, (ii) transfecting cells growing in individual vessels with a dilution of the in vitro recombination reaction mixture from (i) such that a number of such individual vessels contain a single cell that is infected by a recombinant adenovirus comprising the nucleotide sequence encoding the heterologous gene of interest, and (iii) identifying those individual vessels in which a single cell has been infected by the recombinant adenovirus comprising the nucleotide sequence encoding the heterologous gene of interest.

The methods of the first aspect can be advantageously used to produce recombinant adenoviruses for use as vaccines production times reduced from approximately 33-44 weeks down to as little as 28 days.

Using current methods a virus stock is produced by amplifying a bulk transfection that may contain many minor species at extremely low levels that are difficult to detect. Accordingly, three rounds of cloning are required to ensure a clonal stock is produced using such methods. The method of the present invention begins with a characterised viral genome and therefore only the recombinant antigen sequence may be incorrect after recombination and transfection. The transfection is carried out so that only one recombinant viral genome transfects each vessel and therefore there cannot be a mix including many minor species. In very rare cases two viral genomes may transfect the same cell, but if they are not identical in the recombinant antigen coding sequence they can be easily distinguished by sequencing the coding DNA sequence since each viral species will make up around 50% of the mixture and we are no longer looking for minor species. Any virus samples appearing to contain a mixture of correct and incorrect sequence will be discarded and only those that are correct will be selected for use as a vaccine.

Synthetic DNA encoding a heterologous gene of interest may contain minor species which are not completely correct. The method of the invention resolves this problem by producing virus clones immediately after transfection and thus allowing the gene coding sequence in each clone to be sequenced and only correct clones selected. This is advantageous over amplifying a bulk virus stock that potentially represents a mixture of recombinant viruses and then cloning at a later time.

In addition to providing instant cloning and fast expansion of the recombinant adenovirus, the methods of the invention provide an important improvement in repositioning large amounts of quality control (QC) testing necessary for using a recombinant virus as a vaccine to a point before the manufacturing of any specific recombinant adenovirus begins. Such QC testing can be carried out on bulk starting materials, and this offers a considerable time saving when the method is used to generate a recombinant adenovirus for use as a vaccine.

Another advantage of the new method is that it can be used efficiently to generate simian adenoviral vectors as shown herein. Most previous work on rapid adenoviral vector generation has used only one or very few serotypes of human adenovirus, especially human adenovirus serotype 5 (Ad-5). Simian adenoviruses are now preferred over human adenoviruses as vectors for immunisation because i) the are far less negatively impacted by pre-existing anti-vector immunity caused by natural exposure to human adenoviruses; ii) they have been found to be safe and immunogenic in many thousands of subjects (Ewer at al. supra), in contrast to the common human adenovirus vector (Ad-5) which was associated with a major safety signal and concern about enhanced HIV infection in the major “STEP” trial of a Merck HIV vaccine (Cohen (2007) Science 318:28-29).

In a second aspect, the invention provides a composition that comprises an adenoviral genome in which the E1 gene is replaced by an expression cassette comprising a DNA sequence encoding a fluorescent marker protein flanked by a first pair of unique restriction sites not present anywhere else in the adenoviral genome for use in a method of the first aspect.

The compositions of the second aspect can be advantageously used in the methods of the first aspect to allow clear identification of a recombinant adenovirus comprising a nucleotide sequence encoding a heterologous gene of interest. All of the clear advantages of using the method of the first aspect can be found also in the composition of the second aspect.

### BRIEF DESCRIPTION OF THE DRAWINGS

The present invention will now be described by way of example, with reference to the accompanying drawings, in which:

FIG. 1 shows a schematic outline with timings for rapid production of a recombinant adenovirus for use as a vaccine using current methods. QC—quality control; Amp—amplification; GMP—good manufacturing practice; MVSS—master virus seed stock.

FIG. 2 shows a schematic outline with timings for the methods of the present invention used to produce recombinant adenovirus constructs for use as vaccines.

FIG. 3 shows a schematic illustration of a parent virus composition for use in the method of the present invention. In this case the parent virus is the ChAdOx1-Bi-GMP genome that includes three unique restriction sites (Psil, AsiSI and Rsrll) for the insertion of antigen or expression cassette. LPTOS—long tetracycline-regulated CMV promoter.

FIG. 4 shows analysis of TPC-Ad gDNA disrupted with 3M guanidine hydrochloride. 200 ul adenovirus particles (1.4e12VP/ml) were disrupted with 3M guanidine hydrochloride on ice for 45 minutes. 10 μl samples were incubated with (+) or without (−) 2 μg proteinase K at 65° C. for 40 mins and then resolved through 0.7% agarose. M=1 kbp Generuler ladder (Thermofisher).

FIG. 5 shows TPC-Ad gDNA isolation, purification, desalting and filtration after disruption in 3M guanidine hydrochloride and centrifugation on a 2.8MCsCl gradient at 68000 rpm for 18 hrs. DNA was isolated was desalted into 10 mM Tris pH7.8 and filtered through a 0.2 μM syringe filter. Aliquots of DNA were resolved through 0.7% agarose. M=1 kbp Generuler (thermofisher).

FIG. 6 shows protein analysis of TPC-Ad gDNA isolated after centrifugation on a 2.8M CsCl gradient. Samples containing 50 ng TPC-Ad gDNA were resolved through an SDS reducing 4-12% Bis-Tris NuPAGE midigel and stained using silver stain.

FIGS. 7A and 7B shows the binding locations of qPCR primers and probe in the ChAdOx1 and ChAdOx2 and ChAd63 adenoviral genomes.

FIG. 8 shows the in vitro recombination reaction scheme of the claimed method to produce a recombinant ChAdOx1 using ChAdOx1-Bi-GFP as the parental adenoviral genomic DNA.

FIG. 9 shows % cells expressing mCherry and GFP 30h post transfection after transfection with recombination reactions containing various amounts TPC-Ad gDNA and mCherry ORF PCR product. 60, 40, and 20 ng PsiI digested TPC-Ad gDNA were recombined with 40, 20 and 10 ng mCherry ORF PCR product using NEBuilder. Recombination reactions were incubated at 50° C. for 40 minutes then 20° C. for 2 minutes before transfection. T-Rex-293 cells seeded into 96wp were transfected with the recombination reactions using lipofectamine 2000 at a ratio of 1:5. Media containing tetracycline was added 5h after transfection. The number of cells expressing GFP and mCherry was determined 30h post infection by FACS analysis.

FIG. 10 shows an overview of the process for rapid generation of recombinant adenoviruses for use as vaccines.

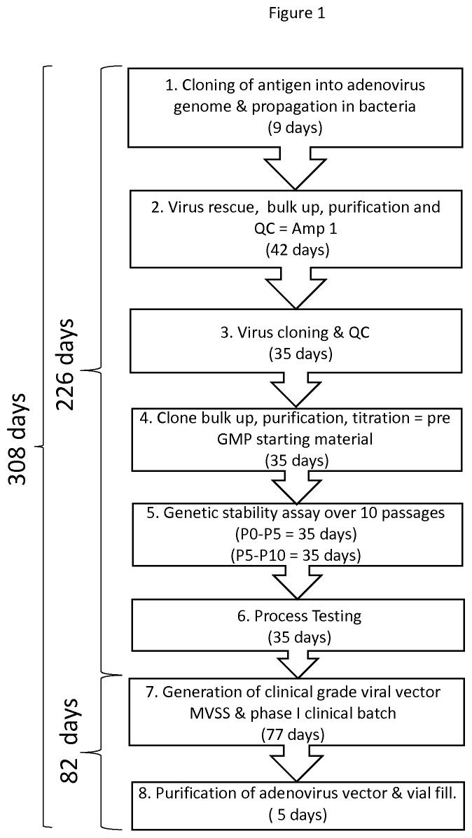

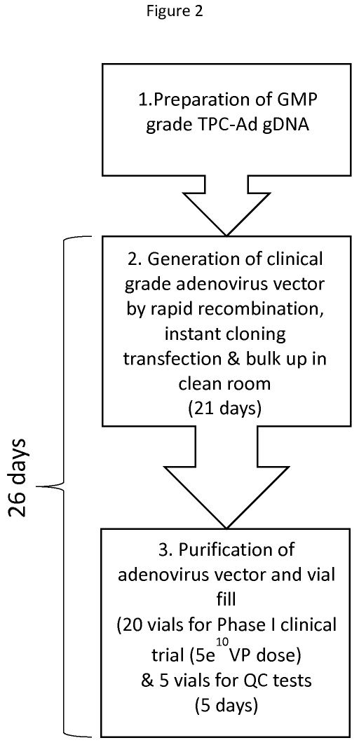

### DETAILED DESCRIPTION

In a first aspect, the invention provides a method for generating a recombinant adenovirus comprising a nucleotide sequence encoding a heterologous gene of interest for use as a vaccine comprising the steps of: (i) inserting the heterologous gene of interest into the adenovirus genome by recombining terminal protein complexed adenovirus genomic DNA (TPC-Ad gDNA) with a synthetic DNA comprising a nucleotide sequence encoding the gene of interest and having at least 15 bp at its 5′ end and at least 15 bp at its 3′ end that are homologous to the insertion site sequence of the adenovirus genomic DNA in an in vitro recombination reaction, (ii) transfecting cells growing in individual vessels with a dilution of the in vitro recombination reaction mixture from (i) such that a number of such individual vessels contain a single cell that is infected by a recombinant adenovirus comprising the nucleotide sequence encoding the heterologous gene of interest, and (iii) identifying those individual vessels in which a single cell has been infected by the recombinant adenovirus comprising the nucleotide sequence encoding the heterologous gene of interest.

The prior art provides a number of methods for producing recombinant adenoviruses, for example Hillgenberg et al. (2006), Choi et al. (2012) and Miciak et al. (2018) amongst others have each provided elegant methods. However none of the methods provided have addressed the need for a protracted cloning process to isolate a recombinant clonal adenovirus for use as a vaccine. The method of the present invention advantageously provides a means of eliminating the protracted cloning process when generating a recombinant adenovirus. This in turn removes a large delay in the generation of recombinant adenoviral vectors, which allows such vectors to be more readily adapted for use as vaccines and in particular as personalized vaccines in the treatment of cancer or rapid response vaccines for outbreak pathogens.

In preferred embodiments of the invention the TPC-Ad gDNA comprises serotype-matched terminal protein and adenovirus genome. Use of a serotype-matched adenoviral genome and terminal protein allows high efficiency virus rescue after recombination and transfection of cells.

In specific embodiments of the invention, the gene of interest codes for a single epitope, a string of epitopes, a segment of an antigen or a complete antigen protein. Provision of various genes of interest allows for development of improved vaccines to protect or treat patients at risk of developing or suffering from a wide variety of diseases including cancer or outbreak diseases.

In alternative embodiments the polynucleotide is a synthetic DNA molecule, a purified DNA restriction fragment or a polymerase chain reaction (PCR) product. The method allows flexibility in selecting the source of DNA to be used in producing a recombinant adenovirus, and this will result in more rapid completion of the method that is critical when producing a recombinant adenovirus for use as a vaccine to prevent or treat many diseases including cancer or outbreak diseases.

In further alternative embodiments the polynucleotide has between 5 and 50 bp at its 5′ end and between 5 and 50 bp at its 3′ end that are homologous to the insertion site sequence of the adenovirus genomic DNA, or the polynucleotide has between 10 and 20 bp at its 5′ end and between 10 and 20 bp at its 3′ end that are homologous to the insertion site sequence of the adenovirus genomic DNA, or the polynucleotide has 15 bp at its 5′ end and 15 bp at its 3′ end that are homologous to the insertion site sequence of the adenovirus genomic DNA. Provision of polynucleotides having suitable homologous ends allows for efficient recombination with the adenovirus genomic DNA thereby allowing for reliable production of recombinant adenoviruses in in vitro recombination reactions using this method.

In preferred embodiments the insertion site sequence of the adenoviral genomic DNA is located within the E1 locus. Deletion of the E1 gene allows for insertion of heterologous expression cassettes and reliable, high-level expression of an antigen of interest in cells infected by the recombinant virus. This provides advantageous properties for a recombinant adenovirus for use as a vaccine.

In certain embodiments the TPC-Ad gDNA is digested at a unique restriction site within the E1 locus of the adenovirus genomic DNA that is flanked at its 5′ end by the long tetracycline-regulated CMV promoter that drives expression of the gene of interest and at its 3′ end by the bovine growth hormone polyadenylation sequence. Digestion of the adenoviral genomic DNA at a unique restriction site with the E1 locus provides suitable end sequences for recombination with a DNA sequence encoding an antigen of interest while also removing the intact parent adenoviral DNA from any recombination reaction. Advantageously, this allows more efficient recombination with a DNA sequence encoding an antigen of interest and also reduces the number of parental adenoviruses regenerated using the method.

In specific embodiments the in vitro recombination reaction comprises 40 ng digested TPC-Ad gDNA and 44 fmol 3′ and 5′ ends of synthetic DNA encoding the gene of interest. Providing such quantities of reactants allows for optimised recombination and generation of recombinant adenovirus comprising the nucleotide sequence encoding a heterologous gene of interest. Advantageously, this allows for transfection of suitable cells with amounts of recombinant adenoviral genomic DNA that increase generation of individual clones using the method of the invention.

In further specific embodiments the cells to be transfected are seeded in individual vessels at a density of 3.75×10⁵ cells·ml⁻¹ one day before transfection. Seeding of cells at this density improves the efficiency of recombinant adenoviral rescue in the cells.

In further specific embodiments the cells are transfected while growing at approximately 80% confluence in individual vessels. This increases expression of adenoviral early genes and improves the efficiency of recombinant adenoviral rescue in the cells.

In further specific embodiments the cells stably express the tetracycline repressor. Use of such cells, for example T-Rex-293 cells, allows for repression of expression of the gene of interest during virus rescue after transfection. Cells are fragile after transfection, and repression of heterologous gene expression minimises cell death and allows for efficient virus rescue at this step of the method.

In particular embodiments the cells being transfected stably express the tetracycline repressor. Expression of the tetracycline repressor in cells being used to rescue recombinant virus prevents expression of the gene of interest which may be toxic to the cells and therefore increases virus rescue.

In additional specific embodiments the in vitro recombination reaction mixture is diluted in transfection medium and divided equally so as to transfect cells growing in 60 individual vessels. Advantageously, dividing and transfecting each recombination reaction into 60 equal parts delivers individual a recombinant adenovirus into a proportion but not all of the 60 individual vessels. This allows the user to identify a number of individual recombinant adenovirus containing wells while including negative control wells that contain no recombinant adenovirus.

In preferred embodiments transfected cells are frozen and thawed to release cell-associated virus and presence of recombinant adenovirus is identified by quantitative PCR (qPCR) using cell lysate from each well of transfected cells and a set of primers and a probe designed to bind to the left end of the genome downstream of the adenoviral inverted terminal repeat (ITR) and upstream of the insertion site of the gene of interest in a non-coding region. This simplified and accelerated sample extraction and screening process allows for easy and rapid identification of the recombinant adenovirus of interest and rules out the presence or parental adenovirus in the sample.

In additional preferred embodiments the adenovirus genome is derived from a human adenovirus or a simian adenovirus, preferably the human adenovirus is not a serotype 5 human adenovirus. In the most preferred embodiments the simian adenovirus is a chimpanzee adenovirus such as ChAdOx1 (Antrobus et al. (2014) Mol Ther. 22(3):668-674), ChAdOx2 (Morris et al. (2016) Future Virol. 11(9):649-659), ChAd3 or Chad63. Use of human or simian adenoviruses allows use of recombinant adenoviruses produced by the method to be used as vaccines in human subjects. Use of simian adenoviruses, and use of ChAdOx1 or ChAdOx2 in particular, provides an improved vaccine that encounters a lower incidence of pre-existing anti-adenoviral immunity when administered to human subjects.

In specific embodiments the individual vessels are separate wells in a multiwell plate. The use of such small volume vessels allows for rapid, economical and efficient transfection of cells and screening of resulting recombinant adenoviruses. The use of multiwall format plates also allows for automation of the method and all the related processes.

In preferred embodiments the TPC-Ad gDNA is provided from a stock of TPC-Ad gDNA material that has undergone and passed requisite quality control (QC) assays allowing use in good manufacturing practice (GMP) biomanufacture. Use of TPC-Ad gDNA from a QC controlled stock of material allows for enhanced rapidity in the method for producing recombinant adenovirus for use as a vaccine. By performing QC testing prior to beginning this method it is possible to pre-test viral components and additionally reduce the testing and assay burden during the adenovirus production and manufacturing process.

In certain embodiments the recombinant adenovirus produced by the method according to the first aspect of the invention can be used as a vaccine to prevent and/or treat diseases in humans or in animals. In particular, recombinant adenovirus produced by the method are very useful in the generation of personalised vaccines for the treatment of cancer. The invention overcomes a major obstacle in providing such treatments using viral vectors: that is the slow time course of generating and developing virally vectored vaccines. The rapid generation of recombinant adenovirus by the new instant method disclosed herein allows sufficient time for the clinical evaluation of a patient, the identification of the patient's own cancer-specific antigen, and the generation of the appropriate recombinant adenovirus vaccine to treat that individual patient. This has not been possible before the development of the claimed method of the first aspect of this invention.

In a second aspect the invention provides a composition that comprises an adenoviral genome in which the E1 gene is replaced by an expression cassette comprising a DNA sequence encoding a fluorescent marker protein flanked by a first pair of unique restriction sites not present anywhere else in the adenoviral genome for use in a method of the first aspect of the invention.

As discussed above, the prior art provides a number of methods for producing recombinant adenoviruses. However each of these methods has begun by using an unmodified adenoviral genome as starting material, and each has therefore had to perform elaborate steps in order to produce digested adenoviral genomic DNA suitable for use in an in vitro recombination reaction. The composition provided by the present aspect of the invention overcomes this obstacle and allows for a single step restriction digestion reaction to prepare adenoviral DNA for recombination with an appropriate heterologous nucleic acid molecule. In addition to simplifying the preparation process for adenoviral genomic DNA, use of this composition also allows for large scale preparation of digested genomic DNA produced from a stock of adenovirus which has already been tested to confirm sterility, lack of mycoplasma, identity and genetic stability of that virus, that can be stored in advance so as to streamline the process for generation of recombinant adenoviruses produced entirely in a GMP-compliant manner ready for clinical use on an as-needed basis.

In specific embodiments the adenoviral genome is derived from a human adenovirus or a simian adenovirus, preferably the human adenovirus is not a serotype 5 human adenovirus. In the more preferred embodiments the adenoviral genome is derived from a simian adenovirus, and most preferably the simian adenovirus is a chimpanzee adenovirus such as ChAdOx1 (Antrobus et al. supra), ChAdOx2 (Morris et al. supra), ChAd3 or Chad63. Use of human or simian adenoviruses allows use of recombinant adenoviruses produced by the method to be used as vaccines in human subjects. Use of simian adenoviruses, and use of ChAdOx1 or ChAdOx2 in particular, provides an improved vaccine that encounters a lower incidence of pre-existing anti-adenoviral immunity when administered to human subjects.

In preferred embodiments the fluorescent marker protein is green fluorescent protein (GFP). The presence of a fluorescent marker protein allows for rapid detection of cells infected by intact adenoviruses which have not undergone recombination to express the gene of interest of this aspect, and GFP is a particularly convenient marker protein that can be readily detected directly by fluorescence microscopy or indirectly, for example using anti-GFP antibodies.

In preferred embodiments the first pair of unique restriction sites are selected from PsiI, AsiSi or RsrII sites.

In certain embodiments the expression cassette further comprises the long tetracycline-regulated CMV promoter 5′ to the DNA sequence encoding the fluorescent marker protein and the bovine growth hormone polyadenylation sequence located 3′ to the DNA sequence encoding the fluorescent marker protein, wherein the first pair of restriction sites are located between the long tetracycline-regulated CMV promoter and the DNA sequence encoding the fluorescent marker protein and between the DNA sequence encoding the fluorescent marker protein and the bovine growth hormone polyadenylation sequence. Advantageously, inclusion of the GFP coding sequence in the parental adenoviral genomic DNA can be used as an effective negative control to identify any cells in which parental adenovirus is regenerated in the method of the first aspect of the invention. A simple screening step can eliminate those viruses expressing GFP from consideration when seeking a recombinant adenovirus comprising a nucleotide sequence encoding a heterologous gene of interest for use as a vaccine. Additionally, the presence of GFP can serve as a helpful marker when generating stocks of parental adenoviral genomic DNA for use in the method of the first aspect.

In preferred embodiments the expression cassette further comprises a second pair of unique restriction sites that are different to the first pair of unique restriction sites and are located 5′ to the long tetracycline-regulated CMV promoter and 3′ to the bovine growth hormone polyadenylation sequence. Advantageously, adding a second pair of unique restriction sites allows for removal of the entire GFP expression cassette, and this allows for generation of a recombinant adenovirus comprising a nucleotide sequence encoding a heterologous gene of interest that is to be expressed using a different promoter system. In such cases the desired promoter and polyadenylation sequences could be designed into the synthetic DNA comprising a nucleotide sequence encoding the gene of interest.

In further preferred embodiments the second pair of unique restriction sites are selected from PsiI, AsiSi or RsrII sites.

In additional embodiments the adenoviral genome is further engineered to comprise an additional unique restriction site at the S15/E4 locus. This allows for recombination of a second synthetic DNA comprising a nucleotide sequence encoding the gene of interest into the adenoviral genomic DNA.

In further additional embodiments the additional unique restriction site is selected from PsiI, AsiSi or RsrII sites.

In specific embodiments the adenoviral genome is complexed with a heterologous or non-serotype-matched terminal protein, but in a preferred embodiment the adenoviral genome is complexed with an autologous or serotype-matched terminal protein.

In further specific embodiments the adenoviral genome lacks Gateway recombination sequences.

In preferred embodiments the composition has undergone and passed requisite quality control (QC) assays allowing use in good manufacturing practice (GMP) biomanufacture. Use of material from a QC controlled stock of material allows for enhanced rapidity in a method for producing recombinant adenovirus for use as a vaccine. By performing QC testing prior to beginning this method it is possible to pre-test viral components and additionally reduce the testing and assay burden during the adenovirus production and manufacturing process.

In a final aspect the invention provides a recombinant adenoviral vector immunogen comprising any of the compositions of the second aspect of the invention and which expresses a pathogen or tumour epitope or antigen to which an immune response is generated in a mammal.

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

### EXAMPLES

### Example 1—Purification of Adenoviral Terminal Protein Complex Viral gDNA (TBC-gDNA) by Caesium Chloride Density Gradient Ultracentrifugation

A 55 kDa terminal protein (TP) is covalently linked to the 5′ end of each strand of adenoviral genomic DNA to produce terminal protein complex viral gDNA (TPC-Ad gDNA). Both serotype matched (“autologous”) and mis-matched (“heterologous”) TPs may be used in the invention. The TP protects the viral gDNA from digestion by cellular exonucleases and acts as a primer for the initiation of DNA replication and forms a heterodimer with DNA polymerase. The TP enhances replication by increasing template activity over 20 fold compared to protein-free templates through subtle changes in the origin of replication allowing binding of other replication factors. The TPC-Ad gDNA is isolated from disrupted purified virus particles using guanidine hydrochloride and purified by caesium chloride density gradient ultracentrifugation.

Purified virus solution containing between 1×10¹¹ and 1×10¹² virus particles (500 μl-1 ml) was aliquoted into a 1.5 ml or 2 ml tube, and an equal volume of filter sterilised 6M Guanidine hydrochloride (GndHCl) made up in nuclease free water was added such that the final GndHCl concentration is 3M. After gentle mixing the diluted virus solution was incubated on ice for 45-60 minutes.

A 2.8M solution of caesium chloride (CsCl) was prepared by adding 9.4281 g CsCl to 20 ml filter sterilised 3M Guanidine hydrochloride (GndHCI) made up in nuclease free water. 2 ml 2.8M CsCl solution was added to an appropriately sized ultracentrifuge tube in a MSCII hood, and the virus/GndHCl solution was gently layered onto the top of the 2.8M CsCl. The virus sample preparation was then centrifuged through the CsCl solution for 18 hours at 68,000 rpm at 20° C. in a Beckman TLA100.3 rotor using the bench Optima TLX ultracentrifuge.

Once the centrifugation was complete TPC-Ad gDNA was removed in 100 μl aliquots and transferred into microfuge tubes. When increased amounts of viral material were present in the starting material a pellet was seen at the conclusion of the CsCl centrifugation step, and it was resuspended in 100 μl 10 mM Tris HCl pH7.8 prepared in nuclease free water. The presence of purified DNA in aliquots removed from the CsCl centrifugation tube was confirmed visually by placing 1 ul of each aliquot on parafilm, adding 1 ul working stock (1:10,000) SYBR safe, and visualising under blue light with orange filter.

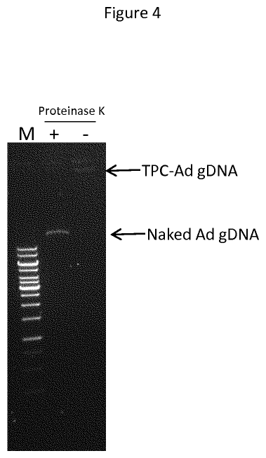

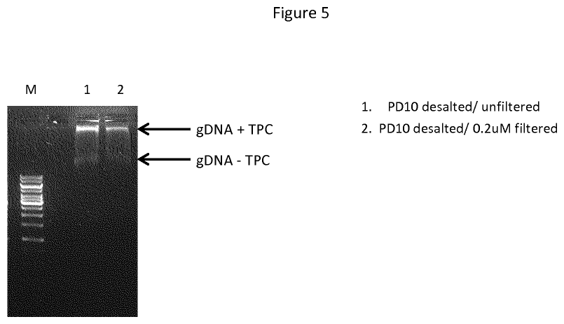

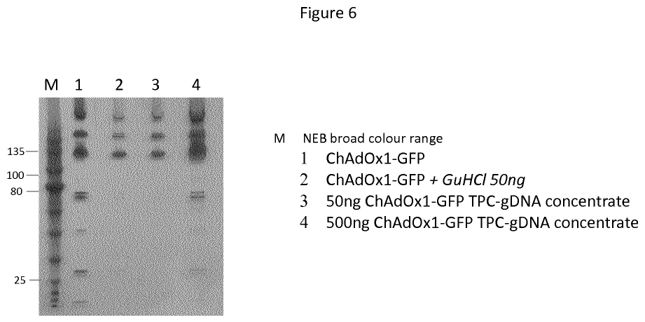

Purified TPC-Ad gDNA from the CsCl centrifugation step was then desalted using a Zeba column equilibrated with 10 mM Tris HCl pH7.8 prepared in nuclease free water. DNA concentrations were determined by spectrophotometry and DNA purity was assessed by gel electrophoresis (FIGS. 4 and 5). Protein levels in TPD-gDNA preparations were examined qualitatively by SDS-PAGE in reducing 4-12% gradient gels (FIG. 6). TPC-Ad gDNA was then stored at −80° C. until needed.

### Example 2—Preparation of TPC-Ad gDNA for Recombination by Digestion with Unique Restriction Enzymes

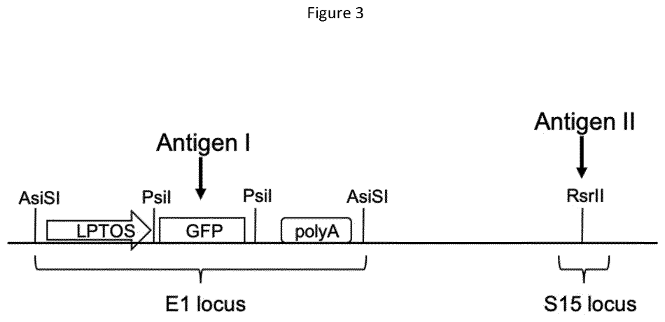

The parental adenoviral genome, for example ChAdOx1-Bi-GFP as shown in FIG. 3, contains the GFP coding sequence at E1 flanked by the long tetracycline-regulated CMV promoter (LPTOS) and Bovine Growth Hormone (BGH) polyadenylation signal (poly A). The GFP ORF is flanked by a pair of unique restriction sites recognized by the PsiI restriction endonuclease and can be excised using PsiI resulting in the generation of 3 fragments: the left arm of the adenoviral genome, the GFP ORF and the right arm of the adenoviral genome. This parental virus can also be digested with AsiSI to excise the complete GFP expression cassette including the LPTOS and poly A. This parental virus can also be digested Rsrll to prepare the gDNA for insertion of an expression cassette at the S14 (E4) locus.

120 ng TPC-Ad gDNA was incubated overnight in an incubator at 37° C. with 10U PsiI in the recommended reaction buffer diluted to a final reaction volume of 30 μl with nuclease free water. The restriction enzyme was inactivated by incubation at 65 C for 20 minutes, and the digested TPC-Ad gDNA was then used directly in recombination reactions once digestion was confirmed by gel electrophoresis or by transfection into cells to confirm that no virus is produced

Example 3—Rapid generation of adenovirus: Recombination and transfection An antigen sequence or expression cassette of interest is introduced into TPC-Ad gDNA by in vitro recombination, and the recombination reaction products are then transfected directly into complementing HEK293 cells for virus rescue. The transfection is performed such that single virus clones are obtained.

NEBuilder (NEB) and In-fusion (Takara) are commercially available systems that allow seamless assembly of multiple DNA fragments, regardless of fragment length or end compatibility. These products can be used for the insertion of antigen/expression cassettes into suitably prepared TPC-Ad gDNA from Example 2. The recombination reaction mix includes exonuclease and polymerase enzymes and in the case of NEBuilder a DNA ligase that work together to produce a double stranded DNA molecule. The exonuclease creates single-stranded 3′ overhangs that facilitate the annealing of fragments that share complementarity at one end (the overlap region) and the polymerase fills in gaps within each annealed fragment. In the NEBuilder reactions the DNA ligase seals nicks in the assembled DNA resulting in a fully sealed DNA molecule rather than relying on the host cell DNA repair machinery to fill in the nicked DNA as is the case for In-fusion reactions.

40 ng PsiI digested TPC-Ad gDNA (10 μl of restriction reaction from example 2) was mixed in a thin-walled PCR tube with 44 fmol of 5′/3′ ends of the required antigen sequence that was synthesised with a minimum 15 bp sequence complementary to the 5′ and 3′ of the TPC-Ad gDNA insertion site. The contents of the tube were collected in the bottom of the tube by briefly spinning in a microfuge. The recombination reaction was then made up to a final volume of 30 μl by addition of the recommended volume of NEBuilder reaction mix or In-fusion reaction mix and nuclease free water. The reaction mixture was incubated at 50° C. for 40 minutes followed by 20° C. for 2 minutes. The recombination reaction was ready for immediate transfection into complimenting cells and rescue of recombinant adenovirus.

For adenovirus rescue the cells need to be dividing to express E1 proteins and support viral replication, and therefore the aim is to achieve approx. 80% confluence on day of transfection. Therefore, T-Rex-293 cells stably expressing the tetracycline repressor were seeded in a 96 well plate 24 hours before transfection at a density of 3.75×10⁴ cells/well in DMEM containing 10% fetal calf serum (FCS) and blasticidin (5 μg/ml).

Pre-warmed Optimem was added to the recombination reaction to achieve a final volume of 100 μl in the reaction tube. 0.5 μl Lipofectamine 2000 per 100 ng DNA in the recombination reaction was added to 100 ml Optimem in a separate tube. Both tubes were incubated at room temperature for 5 minutes. The diluted recombination reaction was then added to the diluted Lipofectamine 2000. The tube was mixed gently before a further 20 minute incubation at room temperature after which the mixture was diluted to a final volume of 3 ml with Optimem.

Media was removed from the T-Rex-293 cells growing in a 96 well plate, and 50 μl of the diluted lipofection reaction was added directly to each one of 60 wells. The cells were then incubated at 37° C. with 8% CO₂ for 4 to 6 hours. The transfection medium in each well was then replaced with 100 ml DMEM containing 10% fetal calf serum (FCS) and blasticidin (5 μg/ml), and the cells are incubated at 37° C. with 8% CO₂.

After 5 days the plates were subjected to 3 freeze/thaw cycles to disrupt the cells and release associated virus. 10 ml of cell lysate from each individual well was removed and DNA isolated from it using the commercially available DNAreleasy reagent analysis by quantitative polymerase chain reaction (QPCR). The remainder of the cell lysate from each well was added to a corresponding well of a 96 well plate seeded 24 hours previously with T-Rex-293 cells at 2.1×10⁴ cells/well in DMEM containing 10% fetal calf serum (FCS) and blasticidin. Recombinant adenovirus was harvested from individual wells when complete cytopathic effects were evident in the wells (between 4 and 6 days after infection).

### Example 4—Quantification of Adenovirus Genome Copy Number by QPCR from Cell Lysate or Purified Virus

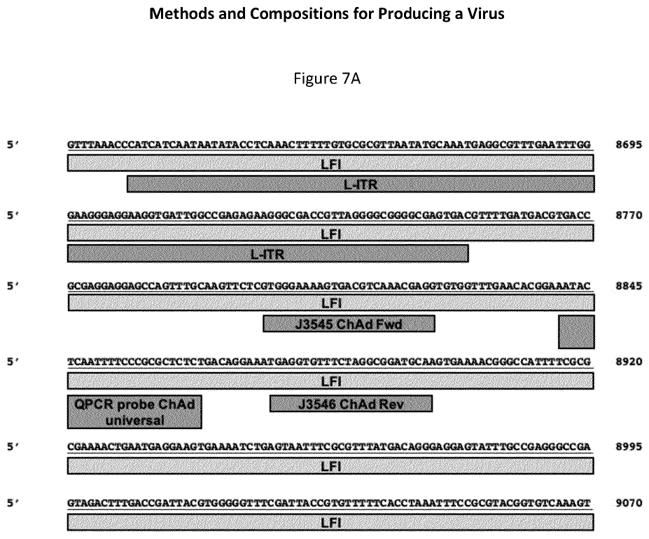

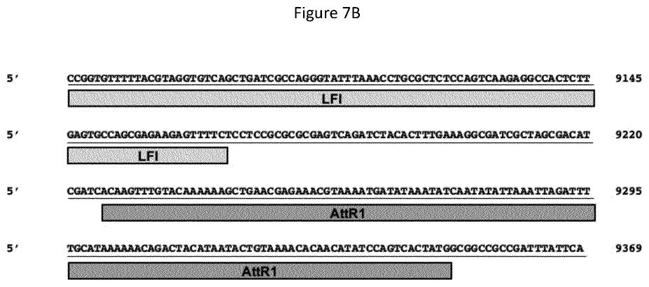

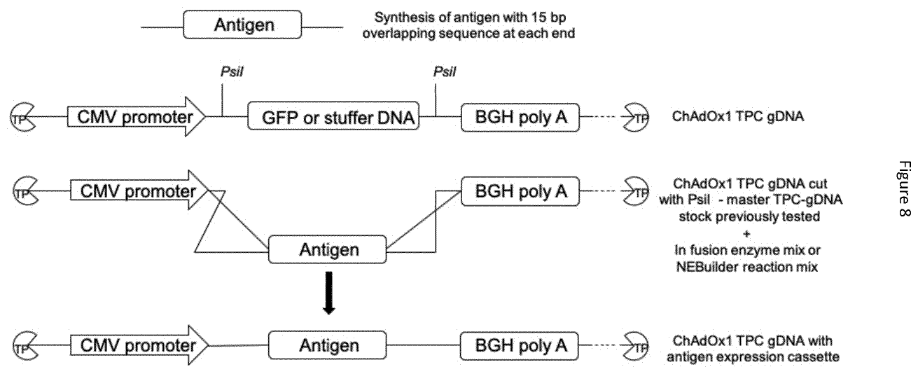

Quantification of ChAdOx1, ChAdOx2 or ChAd63 viral genomes in HEK293 or T-Rex-293 cell lysates is measured by QPCR. The number of viral genomes (which can be related to viral particles on a 1:1 basis) is determined by quantitative PCR (qPCR) from cell lysates processed with DNAReleasy. A set of primers and a probe have been designed that bind to the left end of the genome downstream of the inverted terminal repeat (ITR) and upstream of the antigen insertion region in a non-coding region (see FIGS. 7A and 7B). These primers and probe sequences are:

Primers
ChAd fwd
(SEQ ID NO: 1)
5′GTGGGAAAAGTGACGTCAAACGAG3′
ChAd rev
(SEQ ID NO: 2)
5′TGCATCCGCCTAGAAACACCTCA3′
Probe
ChAd universal probe
(SEQ ID NO: 3)
5′GAGAGCGCGGGAAAATTGAGTATT3′

There is a single mismatch in the reverse primer in ChAdOx2; the sequence of the relevant regions in AdCh63 is identical to that in ChAdOx2 so this method may also be successful for AdCh63. The relevant sequence is not present in AdHu5 vectors.

Cells and media were harvested from a flask showing complete cytopathic effect (CPE). For small volumes cells and media can be harvested directly, but for larger volumes the cells were collected by centrifugation 1500 g for 5 mins and resuspended in 1/10 media volume adenovirus lysis buffer (50 mMTris, 2 mM MgCl₂, pH 9.0). Cells to be harvested are then frozen and thawed three times. 10 μl of lysate was added to 15 μl DNAReleasy reagent, and the sample was processed in a thermocycler using the following cycles: 65° C. for 15 mins, 96° C. for 2 mins, 65° C. for 4 mins, 96° C. for 1 mins, 65° C. for 1 mins, 96° C. for 30 secs, 20° C. hold. Sample was diluted to a total volume of 1 ml and 5 μl was used per QPCR reaction.

QPCR reactions were carried out by initial hotstart activation at 95° C. for 10 minutes followed by 45 cycles of denaturation at 95° C. for 15 seconds followed by denaturation and annealing at 60° C. for 1 minute.

A standard curve is established using the pTOPO-ChAdOx1 LF1 plasmid that is 4,118 base pairs in length and gives the determined genome copy number per ng DNA as shown in Table 1.

### Example 5—Generation of Recombinant ChAdOx1 Expressing mCherry

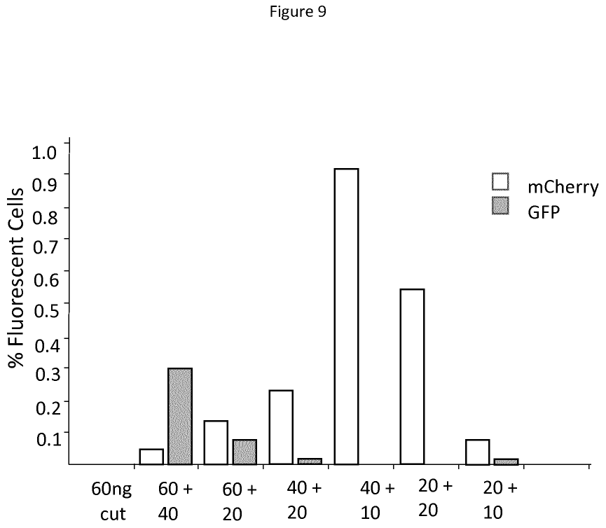

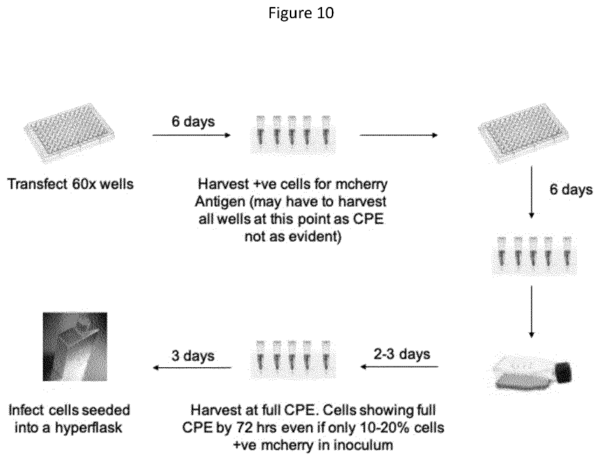

mCherry gene was used as a model antigen for insertion into ChAdOx1. The mCherry gene was amplified using primers containing 15 bp homology to the PsiI insertion site of the TPC-AdgDNA. The TPC-gDNA was digested with PsiI and then the enzyme was heat inactivated prior to recombination. The recombination efficiency of a range of TPC-gDNA and mcherry ORF concentrations using NEBuilder and In-fusion were tested. Reactions were incubated at 50° C. for 40 minutes followed by 2 minutes at 20° C. and then immediately transfected into T-Rex-293 cells seeded into a 96 well plate using lipofectamine 2000 at a ratio of 1:5. The number of GFP (from undigested TPC-Ad gDNA) and mCherry cells (from recombination reactions) 30h post transfection were determined by FACS (FIG. 9).

TABLE 1
Adenoviral genome copy number standard

| Amount of DNA std | Copy number |
|---|---|
| 5 ng | 1 × 10⁹ |
| 0.5 ng | 1 × 10⁸ |
| 50 pg | 1 × 10⁷ |
| 5 pg | 1 × 10⁶ |
| 0.5 pg | 1 × 10⁵ |
| 0.05 pg | 1 × 10⁴ |

What is claimed:

1. A method for generating a recombinant adenovirus comprising a nucleotide sequence encoding a heterologous gene of interest for use as a vaccine comprising the steps of:
i. inserting the heterologous gene of interest into an adenovirus genome by recombining terminal protein complexed adenovirus genomic DNA (TPC-Ad gDNA) with a polynucleotide comprising a nucleotide sequence encoding the gene of interest and having 5′ and 3′ ends that are homologous to an insertion site sequence of the adenovirus genomic DNA in an in vitro recombination reaction,
ii. transfecting cells growing in individual vessels with a dilution of the in vitro recombination reaction mixture from (i) such that a number of such individual vessels contain a single cell that is infected by the recombinant adenovirus comprising the nucleotide sequence encoding the heterologous gene of interest, and
iii. identifying those individual vessels in which a single cell has been infected by the recombinant adenovirus comprising the nucleotide sequence encoding the heterologous gene of interest.

2. A method according to claim 1, wherein the TPC-Ad gDNA comprises a serotype-matched terminal protein and adenovirus genome.

3. A method according to claim 1, wherein the heterologous gene of interest codes for a single epitope, a string of epitopes, a segment of an antigen or a complete antigen protein.

4. A method according to claim 1, wherein the polynucleotide is a synthetic DNA molecule, a purified DNA restriction fragment or a polymerase chain reaction (PCR) product.

5. A method according to claim 1, wherein the polynucleotide has between 5 and 50 bp at its 5′ end and between 5 and 50 bp at its 3′ end that are homologous to the insertion site sequence of the adenovirus genomic DNA.

6. A method according to claim 5, wherein the polynucleotide has between 10 and 20 bp at its 5′ end and between 10 and 20 bp at its 3′ end that are homologous to the insertion site sequence of the adenovirus genomic DNA.

7. A method according to claim 6, wherein the polynucleotide has 15 bp at its 5′ end and 15 bp at its 3′ end that are homologous to the insertion site sequence of the adenovirus genomic DNA.

8. A method according to claim 1, wherein the insertion site sequence of the adenovirus genomic DNA is located within the E1 locus.

9. A method according to claim 1, wherein the TPC-Ad gDNA is digested at a unique restriction site within the insertion site sequence of the adenovirus genomic DNA that is flanked at its 5′ end by a long tetracycline-regulated CMV promoter that drives expression of the gene of interest and at its 3′ end by a bovine growth hormone polyadenylation sequence.

10. A method according to claim 1, wherein the in vitro recombination reaction comprises 40 ng digested TPC-Ad gDNA and 44 fmol 3′ and 5′ ends of the polynucleotide encoding the gene of interest, wherein the polynucleotide encoding the gene of interest is a synthetic DNA.

11. A method according to claim 1, wherein the cells to be transfected are seeded in the individual vessels at a density of 3.75×10⁵ cells·ml⁻¹ one day before transfection.

12. A method according to claim 1, wherein the cells are transfected while growing at approximately 80% confluence in individual vessels.

13. A method according to claim 1, wherein the cells being transfected stably express a tetracycline repressor.

14. A method according to claim 1, wherein the in vitro recombination reaction mixture is diluted in a transfection medium and divided equally so as to transfect cells growing in 60 individual vessels.

15. A method according to claim 1, wherein the transfected cells are frozen and thawed to release cell-associated virus and presence of recombinant adenovirus is identified by quantitative PCR (qPCR) using cell lysate from each well of transfected cells and a set of primers and a probe designed to bind to the left end of the genome downstream of the adenoviral inverted terminal repeat (ITR) and upstream of the insertion site of the gene of interest in a non-coding region.

16. A method according to claim 1, wherein the adenovirus genome is derived from a simian adenovirus.

17. A method according to claim 16, wherein the simian adenovirus is ChAdOx1, ChAdOx2, ChAd3, or ChAd63.

18. A method according to claim 1, wherein the adenovirus genome is derived from a human adenovirus.

19. A method according to claim 18, wherein the human adenovirus is not a serotype 5 human adenovirus.

20. A method according to claim 1, wherein the individual vessels are separate wells in a multiwell plate.

21. A method according to claim 1, wherein the TPC-Ad gDNA is provided from a stock of TPC-Ad gDNA material that has undergone and passed requisite quality control assays allowing use in good manufacturing practice (GMP) biomanufacture.

22. A recombinant adenovirus produced by the method of claim 1.

23. A composition comprising an adenoviral genome in which an E1 gene of the adenoviral genome is replaced by an expression cassette comprising a DNA sequence encoding a fluorescent marker protein flanked by a first pair of unique restriction sites not present anywhere else in the adenoviral genome.

24. A composition according to claim 23, wherein the adenoviral genome is derived from a simian adenovirus.

25. A composition according to claim 24, wherein the simian adenovirus is ChAdOx1, ChAdOx2, ChAd3, or ChAd63.

26. A composition according to claim 23, wherein the adenovirus genome is derived from a human adenovirus.

27. A composition according to claim 26, wherein the human adenovirus is not a serotype 5 human adenovirus.

28. A composition according to claim 23, wherein the fluorescent marker protein is a green fluorescent protein (GFP).

29. A composition according to claim 23, wherein the first pair of unique restriction sites are PsiI, AsiSi or RsrII sites.

30. A composition according to claim 23, wherein the expression cassette further comprises a long tetracycline-regulated CMV promoter 5′ to the DNA sequence encoding the fluorescent marker protein and a bovine growth hormone polyadenylation sequence located 3′ to the DNA sequence encoding the fluorescent marker protein, wherein the first pair of unique restriction sites are located between the long tetracycline-regulated CMV promoter and the DNA sequence encoding the fluorescent marker protein and between the DNA sequence encoding the fluorescent marker protein and the bovine growth hormone polyadenylation sequence.

31. A composition according to claim 30, wherein the expression cassette further comprises a second pair of unique restriction sites that are different to the first pair of unique restriction sites and are located 5′ to the long tetracycline-regulated CMV promoter and 3′ to the bovine growth hormone polyadenylation sequence.

32. A composition according to claim 31, wherein second pair of unique restriction sites are selected from PsiI, AsiSi or RsrII sites.

33. A composition according to claim 23, wherein the adenoviral genome further comprises an additional unique restriction site at an S15/E4 locus.

34. A composition according to claim 33, wherein the additional unique restriction site is an PsiI, AsiSi or RsrII sites.

35. A composition according to claim 23, wherein the adenoviral genome is complexed with an autologous terminal protein.

36. A composition according to claim 23, wherein the adenoviral genome is complexed with a heterologous terminal protein.

37. A composition according to claim 23, wherein the adenoviral genome lacks a Gateway recombination sequence.

38. A composition according to claim 23 that has undergone and passed requisite quality control assays allowing use in good manufacturing practice (GMP) biomanufacture.

39. A recombinant adenoviral vector immunogen comprising a compositions of claim 23 which expresses a pathogen epitope, a tumour epitope, or antigen to which an immune response is generated in a mammal.


SEQUENCE LISTING (3 sequences)

SEQ ID NO: 1 - DNA, 24 bp, Artificial Sequence
  Primer
gtgggaaaagtgacgtcaaacgag

SEQ ID NO: 2 - DNA, 23 bp, Artificial Sequence
  Primer
tgcatccgcctagaaacacctca

SEQ ID NO: 3 - DNA, 24 bp, Artificial Sequence
  Primer/Probe
gagagcgcgggaaaattgagtatt